(19) United States
(12) Patent Application Publication
(10) Pub. No.: US 2026/0325243 A1
(43) Pub. Date: Oct. 22, 2026

(54) TELESCOPIC LINEAR SLIDE MECHANISM FOR A ROBOTIC SURGICAL SYSTEM

(71) Applicant: Covidien LP, Mansfield, MA (US)

(72) Inventors: Ranjan Kumar Mishra, Orange, CT (US); Richard S. Lech, Hamden, CT (US); Ulrich Hagn, Muenchen (DE); Mark H. MacLeod, Middlebury, CT (US); Michael A. Zemlok, Prospect, CT (US)

(21) Appl. No.: 19/649,390

(22) Filed: Apr. 16, 2026

Related U.S. Application Data
(60) Provisional application No. 63/790,244, filed on Apr. 17, 2025.

(51) Int. Cl. A61B 34/35

(52) U.S. Cl. CPC A61B 34/35

(57) ABSTRACT

A linear slide mechanism includes a rail having a linear encoder scale extending linearly along a first side of the rail; a shuttle slidably connected to the first side of the rail so as to translate axially relative to the rail; and a rigid flex printed circuit board (PCB) assembly including an electrically conductive elongate body. The elongate body has a first elongate segment; a second elongate segment overlying the first elongate segment; and an encoder read head connected to the second elongate segment. The encoder read head is operatively connected to the shuttle and is in operative registration with the linear encoder scale. The elongate body of the rigid flex printed circuit board (PCB) assembly constitutes a bi-stable member.

### CROSS REFERENCE TO RELATED APPLICATION

This application claims priority to and the benefit of U.S. Provisional Application Ser. No. 63/790,244 filed on Apr. 17, 2025, the contents of which are incorporated herein by reference.

### BACKGROUND

Robotic surgical systems have been used in minimally invasive medical procedures in which surgical instruments are inserted through surgical ports at fixed entry points into a patient's body. These robotic surgical systems may use a linear prismatic joint or slide on which is supported an instrument drive unit (“IDU”) configured to actuate a surgical instrument connected to the IDU. During use, knowing a relative position of the sliding/telescoping components of the linear prismatic joint or slide is desirable in order to place the surgical instrument at target locations or depths inside a patient. The linear prismatic joint or slide requires electrical communication between the moving components thereof. Cable trays, spring-loaded spiral cable spool type systems are some methods used to establish electrical communication between the moving components in a telescopic linear slide mechanism. However, these systems and methods have their restrictions and issues including additional weight, complexity, size, and reliability. Accordingly, a need exists for a more economical, reliable, and cost-effective system and method for establishing electrical communication between the moving components, for example, a linear prismatic joint or slide.

### SUMMARY

According to aspects, this disclosure is directed to a linear slide mechanism. The linear slide mechanism includes a rail having a linear encoder scale extending linearly along a first side of the rail; a shuttle slidably connected to the first side of the rail so as to translate axially relative to the rail; and a rigid flex printed circuit board (PCB) assembly including an electrically conductive elongate body. The elongate body has a first elongate segment; a second elongate segment overlying the first elongate segment; and an encoder read head connected to the second elongate segment. The encoder read head is operatively connected to the shuttle and is in operative registration with the linear encoder scale. The elongate body of the rigid flex printed circuit board (PCB) assembly constitutes a bi-stable member.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the aspects given below, explain the principles of the disclosure, wherein:

FIG. 1 is a schematic view of a robotic surgical system in accordance with the principles of the present disclosure;

FIG. 2 is a perspective view of a robotic arm of the robotic surgical system of FIG. 1, the robotic arm shown with a surgical assembly coupled thereto, the surgical assembly having a surgical instrument, a linear slide mechanism, and a surgical portal coupled thereto;

FIG. 3 is an enlarged, perspective view of the surgical assembly of FIG. 2 with a different surgical instrument coupled thereto and with the linear slide mechanism thereof shown in a first, extended position;

FIG. 4 is another view of FIG. 3 with the linear slide mechanism shown in a second, retracted position;

FIG. 5 is a perspective view of inner components of the linear slide mechanism of FIGS. 2-4, illustrating the linear slide mechanism in the retracted position;

FIG. 6 is a further perspective view of the inner components of the linear slide mechanism of FIG. 5;

FIG. 7 is a perspective view of the inner components of the linear slide mechanism of FIGS. 2-6, illustrating the linear slide mechanism in the extended position;

FIG. 8 is a cross-sectional view of the linear slide mechanism of FIG. 7, as taken through 8-8 of FIG. 7;

FIG. 9 is a perspective view of rigid-flex printed circuit board assembly with a bi-stable flex member of the linear slide mechanism of FIGS. 2-8;

FIG. 10 is a schematic, perspective illustration of a portion of the bi-stable flex member of FIG. 9; and

FIG. 11 is a cross-sectional view of the bi-stable flex member of FIG. 10, as taken through 11-11 of FIG. 10.

### DETAILED DESCRIPTION

Aspects of this disclosure are described in detail with reference to the drawings, in which like reference numerals designate identical or corresponding elements in each of the several views. As used herein, the term “distal” refers to that portion of a device that is farther from the user, while the term “proximal” refers to that portion of a device that is closer to the user. As used herein, the term “clinician” refers to a doctor, nurse, or other care provider and may include support personnel.

In the following description, well-known functions or constructions are not described in detail to avoid obscuring the present disclosure in unnecessary detail.

Robotic surgical systems have been used in minimally invasive medical procedures and can include robotic arm assemblies. Such procedures may be referred to as what is commonly referred to as “Telesurgery.” Some robotic arm assemblies include one or more robot arms to which surgical instruments can be coupled. These surgical instruments include, for example, electrosurgical forceps, cutting instruments, staplers, graspers, electrocautery devices, or any other endoscopic or open surgical devices. Prior to or during use of the robotic surgical system, various surgical instruments can be selected and connected to the robot arms for selectively actuating end effectors of the connected surgical instruments. Such systems employ various robotic elements to assist the clinician and allow remote operation (or partial remote operation) of surgical instrumentation. Various gears, cams, pulleys, electric and mechanical motors, etc. may be employed for this purpose and may be designed with a robotic surgical system to assist the clinician during the course of an operation or treatment. These robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of clinicians may prep the patient for surgery and configure the robotic surgical system with one or more of the instruments disclosed herein while another clinician (or group of clinicians) remotely controls the instruments via the robotic surgical system. As can be appreciated, a highly skilled clinician may perform multiple operations in multiple locations without leaving his/her remote console which can be both economically advantageous and a benefit to the patient or a series of patients.

This disclosure details a light weight, highly reliable, and robust solution for electrical interface between a stationary rail to a linearly sliding rail for a linear slide mechanism of a robotic arm of a robotic surgical system where space is limited.

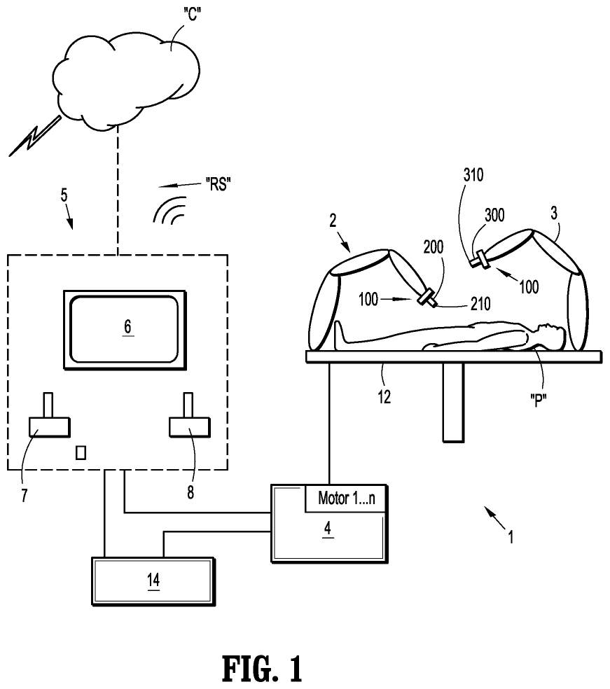

Referring initially to FIG. 1, a surgical system, such as, for example, a robotic surgical system is shown generally as robotic surgical system 1 and generally includes a plurality of robotic arms 2, 3; a controller or control device 4; and an operating console 5 coupled with control device 4. Operating console 5 includes a display device 6, which is set up in particular to display three-dimensional images; and manual input devices 7, 8, by means of which a person (not shown), for example a surgeon, is able to telemanipulate robotic arms 2, 3 in a first operating mode, as known in principle to a person skilled in the art.

Robotic surgical system 1 also includes a surgical assembly 100 connected to a distal end of each of robotic arms 2, 3. Surgical assembly 100 may support one or more surgical instruments such as surgical instruments 200, 300 (e.g., a grasper, stapler, cutter, sealer, or the like).

Each of the robotic arms 2, 3 is composed of a plurality of members, which are connected through joints. Robotic arms 2, 3 may be driven by electric drives (not shown) that are connected to control device 4. Control device 4 (e.g., a computer) is set up to activate the drives, in particular by means of a computer program, in such a way that robotic arms 2, 3, their surgical assemblies 100 and/or surgical instruments 200, 300 execute a desired movement according to a movement defined by means of manual input devices 7, 8. Control device 4 may also be set up in such a way that it regulates movement of robotic arms 2, 3 and/or of the drives. While electrically coupled to controller or control device 4, as described above, robotic arms 2, 3 are configured to receive signals from controller 4, which may be software-based, to establish a remote center of motion at any suitable location.

Robotic surgical system 1 is configured for use on a patient “P” lying on a patient table 12 to be treated in a minimally invasive manner by an end effector of one or more of the surgical instruments. Surgical system 1 may also include more than two robotic arms 2, 3, the additional robotic arms likewise being connected to control device 4 and being telemanipulatable by operating console 5. One or more additional surgical assemblies 100 and/or surgical instruments 200, 300 may also be attached to the additional robotic arm.

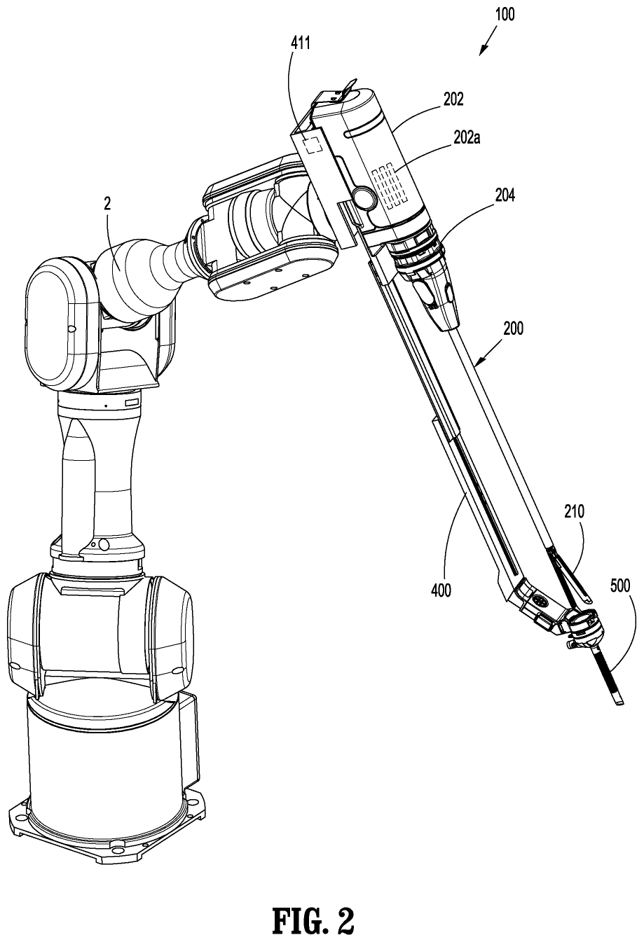

Control device 4 may control a plurality of motors (Motor 1 . . . n) with each motor configured to drive a pushing or a pulling of one or more cables of surgical instruments 200, 300. The plurality of motors can include a plurality of motors 202a of an instrument drive unit 202 that operate surgical instruments 200, 300 through a sterile interface module 204 when a respective one of surgical instruments 200, 300 is connected to sterile interface module 204 as shown in FIG. 2. In use, as these cables are pushed and/or pulled, the one or more cables effect operation and/or movement of end effectors 210, 310 of surgical instruments 200, 300. It is contemplated that control device 4 coordinates the activation of the various motors (Motor 1 . . . n) to coordinate a pushing or a pulling motion of these cables in order to coordinate an operation and/or movement of end effectors 210, 310. In aspects, each motor can be configured to actuate a drive rod or a lever arm to effect operation and/or movement of end effectors 210, 310 in addition to, or instead of one or more cables.

Control device 4 can include any suitable logic control circuit adapted to perform calculations and/or operate according to a set of instructions. Control device 4 can be configured to communicate with a remote system “RS,” either via a wireless (e.g., Wi-Fi, Bluetooth, LTE, etc.) and/or wired connection. Remote system “RS” can include data, instructions and/or information related to the various components, algorithms, and/or operations of workstation 1. Remote system “RS” can include any suitable electronic service, database, platform, cloud “C,” or the like. Control device 4 may include a central processing unit operably connected to memory. The memory may include transitory type memory (e.g., RAM) and/or non-transitory type memory (e.g., flash media, disk media, etc.). In some aspects, the memory is part of, and/or operably coupled to, remote system “RS.”

Control device 4 can include a plurality of inputs and outputs for interfacing with the components of robotic surgical system 1, such as through a driver circuit. Control device 4 can be configured to receive input signals and/or generate output signals to control one or more of the various components (e.g., one or more motors) of robotic surgical system 1. The output signals can include, and/or can be based upon, algorithmic instructions which may be pre-programmed and/or input by a user. Control device 4 can be configured to accept a plurality of user inputs from a user interface (e.g., switches, buttons, touch screen, etc. of operating console 5) which may be coupled to remote system “RS.”

A database 14 can be directly and/or indirectly coupled to control device 4. Database 14 can be configured to store pre-operative data from living being(s) and/or anatomical atlas(es). Database 14 can include memory which can be part of, and/or or operatively coupled to, remote system “RS.”

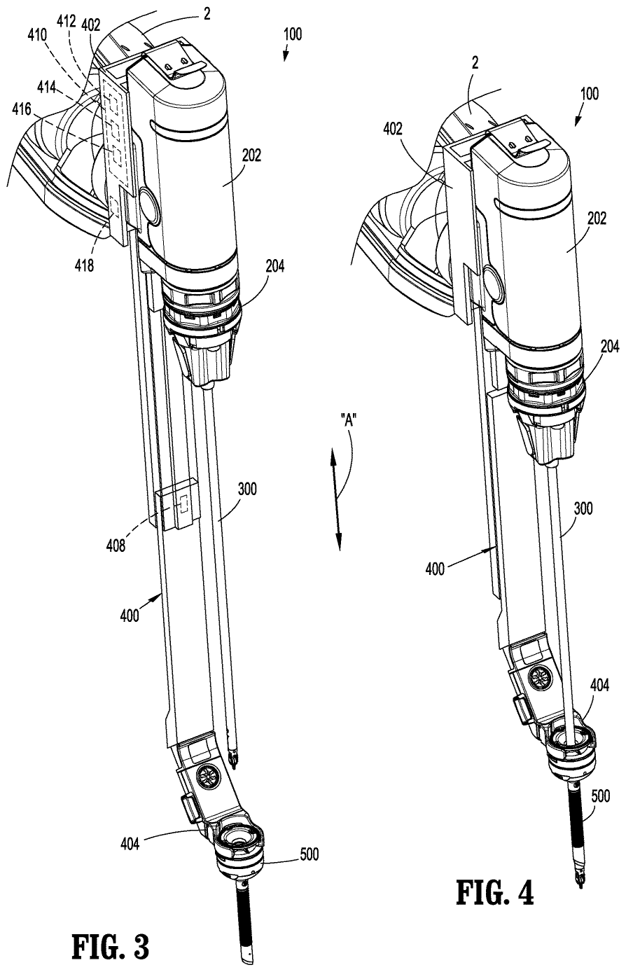

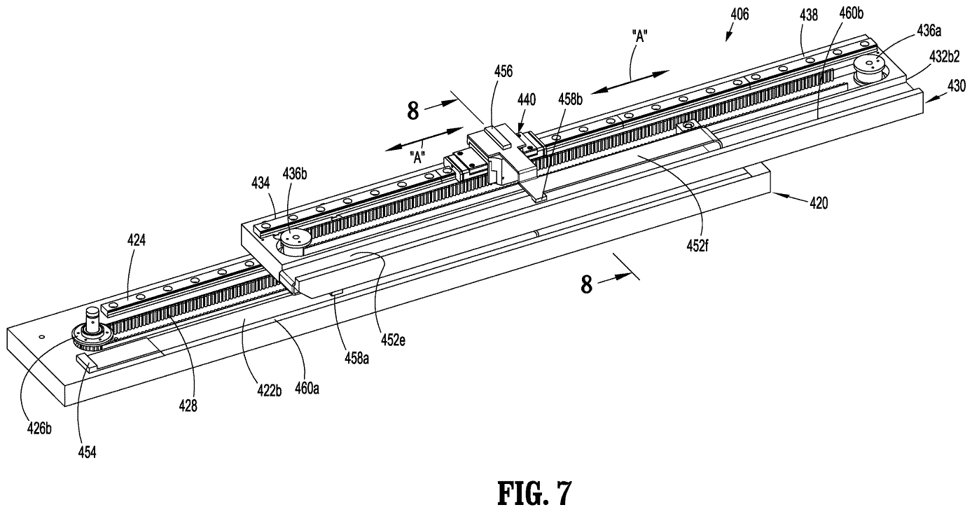

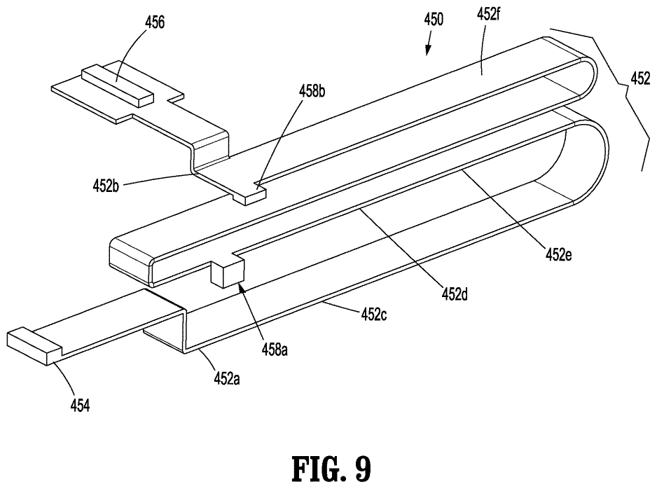

Turning now to FIGS. 2-11, surgical assembly 100 of robotic surgical system 1 includes an instrument drive unit 202, a sterile interface module 204 coupled to instrument drive unit 202, and one or more surgical instruments, such as surgical instrument 300, that are selectively attachable to sterile interface module 204 and actuatable by instrument drive unit 202.

Surgical assembly 100 further includes a linear slide mechanism 400 for actively supporting a surgical port or portal 500 on a distal end portion thereof. Linear slide mechanism 400 can be an active servo joint that provides redundant instrument insertion and retraction.

Linear slide mechanism 400 of surgical assembly 100 includes a drive housing 402 at a proximal end portion thereof which is supported on the distal end of robotic arms 2, 3, and which is configured to support the instrument drive unit 202, and a mount or latch assembly 404 at a distal end portion thereof that is configured to selectively couple to surgical portal 500 for securing surgical portal 500 to linear slide mechanism 400.

Linear slide mechanism 400 further includes an arm or rail assembly 406 that connects latch assembly 404 to drive housing 402 of linear slide mechanism 400. Rail assembly 406 is telescopic and includes a first or stationary rail 420, a second or outer movable rail 430 slidably supported on the first rail 420 for linear translation relative to the first rail 420 (as illustrated by arrows “A”), and a carriage or shuttle 440 slidably supported on the second rail 430 for linear translation relative to the second rail 430 (as illustrated by arrows “A”).

The first rail 420 is secured to the drive housing 402 in a fixed manner whereby movement of the robotic arm 2, 3 results in concomitant movement of the first rail 420, and in turn the linear slide mechanism 400. The first rail 420 includes a first or back side 420a connected to the drive housing 402, and a second or front side 420b opposite the back side 420a. While shown and described as the drive housing 402 being connected to the back side 420a of the first rail 420, it is envisioned that the drive housing 402 may be connected to any side of the first rail 420.

The first rail 420 includes a first or central linearly extending channel 422a formed in the front side 420b of the first rail 420, a second or lateral linearly extending channel 422b formed in the front side 420b of the first rail 420 and offset laterally from the first channel 422a, and a linearly extending rib 424 supported on the front side 420b of the first rail 420 and offset laterally from the first channel 422a along a side opposite the second channel 422b.

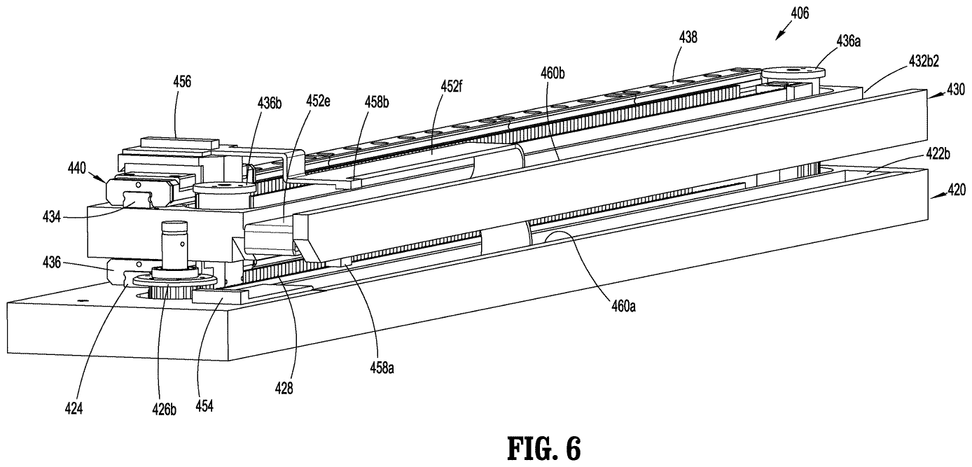

The second rail 430 is slidably connected to the first rail 420 so as to linearly translate or telescope relative to the first rail. The second rail 430 includes a first or back side 430a connected slidably connected to the rib 424 of the first rail 420 via a sliding cuff or connector 436 (see FIG. 6), and a second or front side 430b opposite the back side 430a. While shown and described as the second rail 430 being connected to the front side 420b of the first rail 420, it is envisioned that the second rail 430 may be connected to any side of the first rail 420.

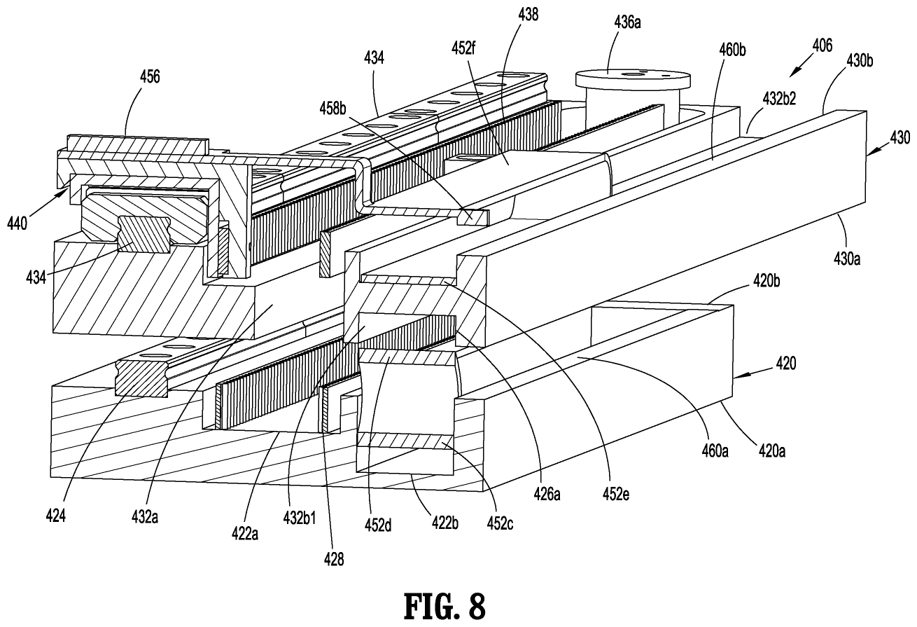

The second rail 430 includes a first or central linearly extending channel 432a formed in the front side 430a of the second rail 430 (or extending completely through the second rail 430), a second or lateral linearly extending channel 432b1 (FIG. 8) formed in the back side 430a of the second rail 430 and offset laterally from the first channel 432a, a third or lateral linearly extending channel 432b2 formed in the front side 430b of the second rail 430 and extending parallel to the second channel 432b1, and a linearly extending rib 434 supported on the front side 430b of the second rail 430 and offset laterally from the first channel 432a along a side opposite the second channels 432b1, 432b2. The linearly extending rib 434 slidably supports the shuttle 440 thereon.

The first rail 420 includes a pair of pulleys, specifically, a first or top pulley 426a (see FIG. 8) located proximate a first or upper end of the first channel 422a, and a second or bottom pulley 426b located proximate a second or lower end of the first channel 422a. The first rail 420 includes a first rail belt 428 extending around and operatively connected to the first pulley 426a and the second pulley 426b of the first rail 420. The sliding connector 436 is secured to the first rail belt 428 so as to move together with the first rail belt 428. It is envisioned that the first pulley 426a and/or the second pulley 426b may be operatively connected to a motor (not shown) or the like, whereby the first pulley 426a and/or the second pulley 426b may be driven and thus translate the first rail belt 428 through the first channel 422a. Since the connector 436 is secured to the first rail belt 428, and since the second rail 430 is secured to the connector 436, in operation, as the first rail belt 428 is actuated, the first rail belt 428 causes the connector 436 to translate along the first rail 420, and in turn, causes the second rail 430 to translate (or telescope) relative to the first rail 420.

The second rail 430 includes a pair of pulleys, specifically, a first or top pulley 436a located proximate a first or upper end of the first channel 432a, and a second or bottom pulley 436b located proximate a second or lower end of the first channel 432a. The second rail 430 includes a second rail belt 438 extending around and operatively connected to the first pulley 436a and the second pulley 436b of the second rail 430. The shuttle 440 is secured to the second rail belt 438 so as to move together with the second rail belt 438. It is envisioned that the first pulley 436a and/or the second pulley 436b may be operatively connected to a motor (not shown) or the like, whereby the first pulley 436a and/or the second pulley 436b may be driven and thus translate the second rail belt 438 through the first channel 432a. Since the shuttle 440 is secured to the second rail belt 438, and since the shuttle 440 is slidably connected to the linearly extending rib 434, in operation, as the second rail belt 438 is actuated, the second rail belt 438 causes the shuttle 440 to translate along the linearly extending rib 434 of the second rail 430, and in turn, causes the driving housing 402 and the instrument drive unit 202 (see FIGS. 1-4) which are each supported or connected to the shuttle 440, to translate along the second rail 430.

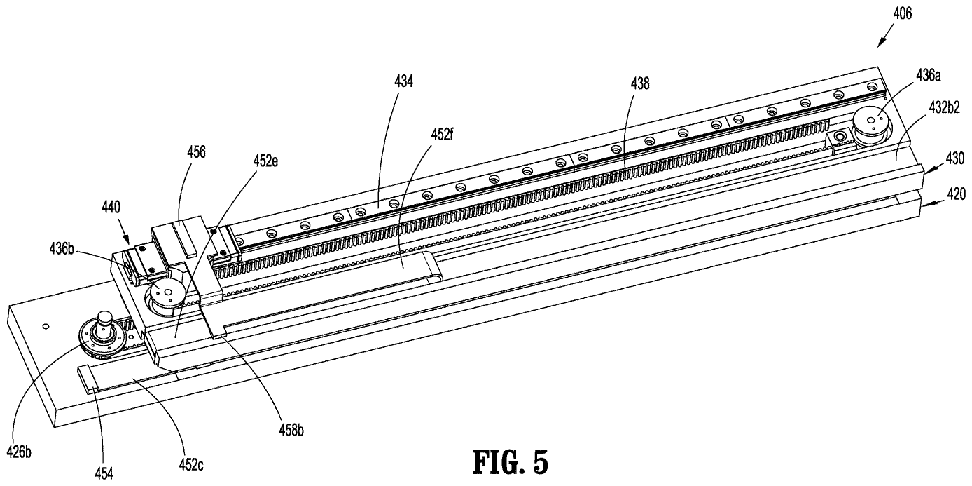

With reference to FIGS. 5-11, the rail assembly 406 includes a rigid flex printed circuit board (PCB) assembly 450 configured to transmit power and data between the control device 4 and the instrument drive unit 202. The rigid flex PCB assembly 450 includes an elongate body or tape portion 452 having a first elongate segment 452c, a second elongate segment 452d, a third elongate segment 452e, and a fourth elongate segment 452f. The elongate body 452 may include wiring or other electrically conductive material therein or thereon for communicating power and/or data along the rigid flex PCB assembly 450 and, for example, from/between the robotic arm 2, 3 and the instrument drive unit 202.

The rigid flex PCB assembly 450 includes a first electrical terminal 454 supported at and connected to a first end 452a of the body portion 452, and more specifically to a first end of the first elongate segment 452c of the body portion 452, as will be described in greater detail below. The rigid flex PCB assembly 450 further includes a second electrical terminal 456 supported at and connected to a second end 452b of the body portion 452, and more specifically to a first end of a fourth elongate segment 452f of the body portion 452, as will be described in greater detail below.

The first elongate segment 452c includes a first end coinciding with the first end 452a of the body portion 452 and which is connected to and supports the first electrical terminal 454. The first elongate segment 452c includes a second end opposite the first end. The first elongate segment 452c may be laid within or received within the second channel 422b of the first rail 420.

The second elongate segment 452d overlies the first elongate segment 452c. The second elongate segment 452d includes a first end connected to and integrally extending from the second end of the first elongate segment 452c. The second elongate segment 452d includes a second end opposite the first end. The second elongate segment 452d may be laid within or received within the second or lateral linearly extending channel 432b1 formed in the back side 430a of the second rail 430.

The third elongate segment 452e overlies the second elongate segment 452d. The third elongate segment 452e includes a first end connected to and integrally extending from the first end of the second elongate segment 452d. The third elongate segment 452e includes a second end opposite the first end. The third elongate segment 452e may be laid within or received within the third or lateral linearly extending channel 432b2 formed in the front side 430b of the second rail 430. In this manner, the second elongate segment 452d and the third elongate segment 452e wrap around a distal end of the second rail 430. In accordance with the present disclosure, the first end of the second elongate segment 452d and/or the first end of the third elongate segment 452e are fixedly secured to the distal end of the second rail 430, wherein axial translation of the second rail 430 relative to the first rail 420 causes the second elongate segment 452d to axially slide or translate relative to the first elongate segment 452c.

The fourth elongate segment 452f overlies the third elongate segment 452e. The fourth elongate segment 452f includes a second end connected to and integrally extending from the second end of the third elongate segment 452e. The fourth elongate segment 452f includes a first end opposite the second end. The fourth elongate segment 452f may also be laid within or received within the third or lateral linearly extending channel 432b2 formed in the front side 430b of the second rail 430.

The rigid flex PCB assembly 450 includes a first encoder read head 458a (e.g., an optical, magnetic, or capacitive linear encoder read head) supported on and extending laterally from the second elongate segment 452d of the body portion 452. The first encoder read head 458a is in operative registration with a first linear encoder scale 460a extending linearly on the first rail 420. It is envisioned that the first encoder read head 458a may be fixedly secured to the second rail 430. In operation, as the second rail 430 axially translates relative to the first rail 420, the first encoder read head 458a translates axial along the first linear encoder scale 460a to thereby read information from the first linear encoder scale 460a and know the actual position of the second rail 430 relative to the first rail 420.

The rigid flex PCB assembly 450 includes a second encoder read head 458b (e.g., an optical, magnetic, or capacitive linear encoder read head) supported on and extending laterally from the fourth elongate segment 452f of the body portion 452. The second encoder read head 458b is in operative registration with a second linear encoder scale 460b extending linearly on the second rail 430. It is envisioned that the second encoder read head 458b may be fixedly secured to the shuttle 440 or the drive housing 402 or the instrument drive unit 202. In operation, as the shuttle 440 axially translates relative to the second rail 430, the second encoder read head 458b translates axial along the second linear encoder scale 460b to thereby read information from the second linear encoder scale 460b and know the actual position of the shuttle 440 relative to the second rail 430.

The relative position between the first encoder read head 458a and the first linear encoder scale 460a, and the relative position between the second encoder read head 458b and the second linear encoder scale 460b must be precise in order to accurately know the location of the second rail 430 relative to the first rail 420 and/or the location of the shuttle 440 relative to the second rail 430. As such, the body portion 452 of the rigid flex PCB assembly 450 is constructed from material or composite of materials that is resistant to axial stretching or shortening, which is resistant to gravitational effects when oriented horizontally, vertically or at an angle therebetween.

In use, the first encoder read head 458a much be in operative registration with the first linear encoder scale 460a, and the second encoder read head 458b must be in operative registration with the second linear encoder scale 460b, whereby the operative registration of the components allows for information and/or data to be communicated or transmitted by and between the components that are in operative registration with one another.

While the rail assembly 406 is shown and described above as having a first or stationary rail 420, a second or outer movable rail 430 slidably supported on the first rail 420 for linear translation relative to the first rail 420, and a carriage or shuttle 440 slidably supported on the second rail 430 for linear translation relative to the second rail 430, it is envisioned and contemplated that the rail assembly 406 may include only a stationary rail 420 and a carriage or shuttle slidably supported on the first rail 420. Accordingly, the rail assembly 406 could be provided with a rigid flex PCB assembly 450 including 454 a first electrical terminal 454 supported at and connected to a first end 452a of the a first elongate segment 452c of the body portion 452 of the rigid flex PCB assembly 450, and a second electrical terminal 456 supported at and connected to a second end 452b of a second elongate segment 452d of the body portion 452 of the rigid flex PCB assembly 450, wherein the a first elongate segment 452c and the second elongate segment 452d overlie one another. The second electrical terminal 456 may be connected to the shuttle 440.

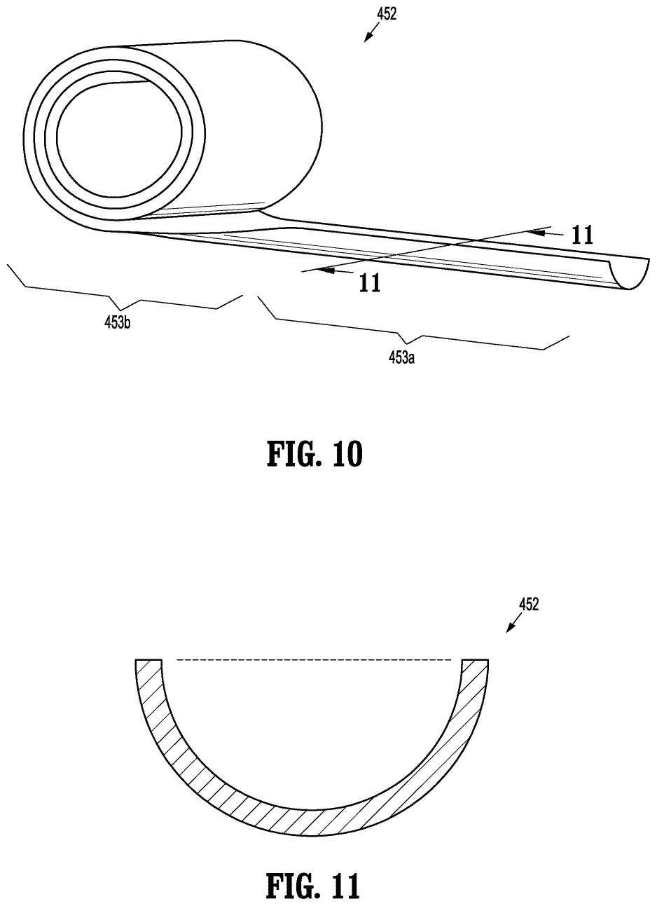

Referring now to FIGS. 10 and 11, a schematic illustration of a segment of the body portion 452 of the rigid flex PCB assembly 450 is shown and will be described. The body portion 452 is formed of a strip of sheet-like material, i.e., the body portion 452 is thin in cross-section. In embodiments, the body portion 452 may be less than 5 mm, or less than 2 mm, or less than 1 mm thin in cross section. In some examples, additional layers may be attached to the body portion 452 for various other purposes, such as providing protection from impact damage. These addition layers may give rise to higher overall thickness in the body portion 452.

As illustrated in FIG. 10, the body portion 452 could have an extended or linear portion 453a and a coiled portion 453b if not contained within channels or guides, such as the second channel 422b of the first rail 420 and/or the second channel 432b1 or the third channel 432b2 of the second rail 430. The second channel 422b of the first rail 420 and the second channel 432b1 of the second rail 430 function to guide the body portion 452 and to inhibit folding over or forceful deforming of the rigid flex PCB assembly 450. The body portion 452 is configured such that it is capable of being progressively transformed between being completely coiled and being completely extended.

As shown in FIG. 11, the extended portion 453a is resiliently biased to have a cross section that is curved in the form of a partial circle. Thus, the extended portion 453b may form a slit tube. As will be appreciated, the curvature of the extended portion 453a can be chosen to be relatively small, but should be large enough to give sufficient stiffness to the extended member 453a to aid in maintaining a linearity thereof. The curved cross-section of the extended portion 453a (e.g., the first elongate segment 452c, the second elongate segment 452d, the third elongate segment 452e, and/or the fourth elongate segment 452f) of the rigid flex PCB assembly 450 may retain its straight rigidness along an entire length of the extended portion 453a. It is contemplated that using a relatively larger curve can be useful in some cases to give additional stiffness to the body portion 452, and resistance to axial lengthening or shortening (as is the case with standard ribbon cables and the like).

The body portion 452, when extended, is resiliently biased in this form so that it provides a stiff substrate for circuits or electrical traces integrated into the body portion 452. Thus, this arrangement preserves the desired degree of linearity, curvature and/or tension in the body portion 452, so that this is optimized for the purpose at hand without any requirement to post tension or locally position the body portion 452, as would be the need for typical ribbon cables or the like.

As so described, the body portion 452 of the rigid flex PCB assembly 450 constitutes a bi-stable member which generally is made from material that creates a bias towards configuring the material in the extended form (e.g. having a curved transverse cross-section in the present disclosure), as well as material that creates a bias opposite to the first bias (e.g. one that biases the member towards its flattened, retracted or coiled form). The member can include a resilient substrate, made of metal for example, which is biased toward the extended form (e.g. biased toward making the member have a semi-or circular-cross-section), laminated or overmolded with a plastic layer that tends to bias the member towards the retracted form (e.g. having a flattened cross-section). Alternatively, the member can include a strip or sheet of a thermoplastic material having prestressing means attached thereto or embedded therein. One particular example is a thermoplastic strip having prestressed fibers therein (such as fibers of glass, carbon, or polymeric materials). The fibers can be located at different angles relative to each other in the plane of the coiled member, such as having one set of fibers that are longitudinally extending and a second set of fibers that are transversely extending.

The body portion 452 of the rigid flex PCB assembly 450 has an inherent bi-stable characteristic to its physical properties by virtue of the body portion 452 having a transverse cross-sectional profile which is curved. In this manner, when the curved portion of the body portion 452 (i.e., the curved portion of the body portion 452 interconnecting the first elongate segment 452c and the second elongate segment 452d) transitions from the first rail 420 to the second rail 430, the body portion 452 bends to a natural bend radius inherent in the particular design of the body portion 452, thereby obviating kinking or crimping of the body portion 452 during use of the rail assembly 406. A minimum bend radius for the curved portion of the body portion 452 is maintained through the spacing or distance between the stationary or first rail 420 and moving member or second rail 430. During use, the curved portion of the body portion 452 shifts along the length of the first rail 420 and/or the second rail 430 as the first rail 420 and the second rail 430 translate relative to one another.

By providing a rigid flex PCB assembly 450 including a body portion 452, as described hereinabove, incidents of kinking or damage to the body portion 452 are reduced due to the ability of the body portion 452 to maintain a minimum bend radius, such as, for example, the first end of the second elongate segment 452d of the body portion 452 that connects to and integrally extends from the second end of the first elongate segment 452c of the body portion 452, and similarly, the first end of the fourth elongate segment 452f of the body portion 452 that connects to and integrally extends from the second end of the third elongate segment 452e of the body portion 452.

Further, a rigid flex PCB assembly 450 including a body portion 452, as described hereinabove, may function as a strain relief, wherein the metal substrate of the body portion 452 is capable of taking relatively high tensile forces or loads when the body portion 452 is in an unspooled condition.

Still further, due to the bi-stable nature of the body portion 452 of the rigid flex PCB assembly 450 and the ability of the body portion 452 to be able to coil or spool up (in the manner of a tape measure), if needed, the body portion 452 provides increased stability to the rigid flex PCB assembly 450 as compared to ribbon cables or cables supported by springs and the like.

The implementation of the rigid flex PCB assembly 450 of the present disclosure allows for the construction of a low profile linear slide mechanism 400, in accordance with the present disclosure, and as described hereinabove.

While a rigid flex PCB assembly 450 has been described herein above, and illustrated in the figures of the present application, it is understood that the rigid flex PCB assembly 450 may be replaced by a rigid printed circuit board assembly (constructed of only rigid material or substantially of rigid material); a rigidized flex printed circuit board assembly (i.e., a flex printed circuit board assembly that has a material attached to a flexible substrate and no electrical contacts through the rigid material); a rigi-flex printed circuit board assembly (i.e., a printed circuit board assembly that has routing layers on both the rigid material and the flex material); a flexible printed circuit board assembly including a rigid substrate on a first side and a second side thereof; or any combinations thereof.

As can be appreciated, securement of any of the components of the disclosed systems can be effectuated using known securement techniques such as welding, crimping, gluing, fastening, etc.

It should be understood that the disclosed structure can include any suitable mechanical, electrical, and/or chemical components for operating the disclosed system or components thereof. For instance, such electrical components can include, for example, any suitable electrical and/or electromechanical, and/or electrochemical circuitry, which may include or be coupled to one or more printed circuit boards. As appreciated, the disclosed computing or control devices (and/or servers), can include, for example, a “controller,” “processor,” “digital processing device” and like terms, and which are used to indicate a microprocessor or central processing unit (CPU). The CPU is the electronic circuitry within a computer that carries out the instructions of a computer program by performing the basic arithmetic, logical, control and input/output (I/O) operations specified by the instructions, and by way of non-limiting examples, include server computers. In some aspects, the controller includes an operating system configured to perform executable instructions. The operating system is, for example, software, including programs and data, which manages hardware of the disclosed apparatus and provides services for execution of applications for use with the disclosed apparatus. In some aspects, the operating system is provided by cloud computing.

In some aspects, the term “controller” may be used to indicate a device that controls the transfer of data from a computer or computing device to a peripheral or separate device and vice versa, and/or a mechanical and/or electromechanical device (e.g., a lever, knob, etc.) that mechanically operates and/or actuates a peripheral or separate device.

In aspects, the controller includes a storage and/or memory device. The storage and/or memory device is one or more physical apparatus used to store data or programs on a temporary or permanent basis. In some aspects, the controller includes volatile memory and requires power to maintain stored information. In various aspects, the controller includes non-volatile memory and retains stored information when it is not powered. In some aspects, the non-volatile memory includes flash memory. In certain aspects, the non-volatile memory includes dynamic random-access memory (DRAM). In some aspects, the non-volatile memory includes ferroelectric random-access memory (FRAM). In various aspects, the non-volatile memory includes phase-change random access memory (PRAM). In certain aspects, the controller is a storage device including, by way of non-limiting examples, CD-ROMs, DVDs, flash memory devices, magnetic disk drives, magnetic tapes drives, optical disk drives, and cloud-computing-based storage. In various aspects, the storage and/or memory device is a combination of devices such as those disclosed herein.

In various aspects, the memory can be random access memory, read-only memory, magnetic disk memory, solid state memory, optical disc memory, and/or another type of memory. In various aspects, the memory can be separate from the controller and can communicate with the processor through communication buses of a circuit board and/or through communication cables such as serial ATA cables or other types of cables. The memory includes computer-readable instructions that are executable by the processor to operate the controller. In various aspects, the controller may include a wireless network interface to communicate with other computers or a server. In aspects, a storage device may be used for storing data. In various aspects, the processor may be, for example, without limitation, a digital signal processor, a microprocessor, an ASIC, a graphics processing unit (“GPU”), field-programmable gate array (“FPGA”), or a central processing unit (“CPU”).

The memory stores suitable instructions and/or applications, to be executed by the processor, for receiving the sensed data (e.g., sensed data from camera), accessing storage device of the controller, generating a raw image based on the sensed data, comparing the raw image to a calibration data set, identifying an object based on the raw image compared to the calibration data set, transmitting object data to a post-processing unit, and displaying the object data to a graphic user interface. Although illustrated as part of the disclosed structure, it is also contemplated that a controller may be remote from the disclosed structure (e.g., on a remote server), and accessible by the disclosed structure via a wired or wireless connection. In aspects where the controller is remote, it is contemplated that the controller may be accessible by, and connected to, multiple structures and/or components of the disclosed system.

The term “application” may include a computer program designed to perform particular functions, tasks, or activities for the benefit of a user. Application may refer to, for example, software running locally or remotely, as a standalone program or in a web browser, or other software which would be understood by one skilled in the art to be an application. An application may run on the disclosed controllers or on a user device, including for example, on a mobile device, an IOT device, or a server system.

As used herein, the controller includes software modules for managing various aspects and functions of the disclosed system or components thereof.

The disclosed structure may also utilize one or more controllers to receive various information and transform the received information to generate an output. The controller may include any type of computing device, computational circuit, or any type of processor or processing circuit capable of executing a series of instructions that are stored in memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, cause the one or more processors to perform one or more methods and/or algorithms.

The phrases “in an aspect,” “in aspects,” “in various aspects,” “in some aspects,” or “in other aspects” may each refer to one or more of the same or different aspects in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques).

Certain aspects of this disclosure may include some, all, or none of the above advantages and/or one or more other advantages readily apparent to those skilled in the art from the drawings, descriptions, and claims included herein. Moreover, while specific advantages have been enumerated above, the various aspects of the present disclosure may include all, some, or none of the enumerated advantages and/or other advantages not specifically enumerated above.

The aspects disclosed herein are examples of the disclosure and may be embodied in various forms. For instance, although certain aspects herein are described as separate, each of the aspects herein may be combined with one or more of the other aspects herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

Further aspects and embodiments of the present disclosure are set out in the below numbered clauses:
1. A robotic surgical system, comprising:
a robotic arm; and
a linear slide mechanism coupled to the robotic arm, the linear slide mechanism including:
   a first rail having a first side and a second side opposite the first side, the second side of the first rail being connected to the robotic arm;
   a second rail having a first side and a second side opposite the first side, the second side of the second rail being slidably connected to the first side of the first rail so as to translate axially relative to the first rail;
   a shuttle slidably connected to the first side of the second rail so as to translate axially relative to the second rail; and
   a rigid flex printed circuit board (PCB) assembly including an electrically conductive elongate body, the elongate body having:
      a first elongate segment having a first end and a second end;
      a second elongate segment having a first end and a second end, the second end of the second elongate segment being connected to the second end of the first elongate segment, wherein the second elongate segment overlies the first elongate segment, wherein the first elongate segment and the second elongate segment are located between the first side of the first rail and the second side of the second rail;
      a third elongate segment having a first end and a second end, the first end of the third elongate segment being connected to the first end of the second elongate segment;
      a fourth elongate segment having a first end and a second end, the second end of the fourth elongate segment being connected to the second end of the third elongate segment, wherein the fourth elongate segment overlies the third elongate segment, wherein the third elongate segment and the fourth elongate segment are located adjacent the first side of the second rail;
      a first electrical connector connected to the first end of the first elongate segment; and
      a second electrical connector connected to the first end of the fourth elongate segment and supported on the shuttle,
      wherein the elongate body of the rigid flex PCB assembly constitutes a bi-stable member.
2. The robotic surgical system of clause 1, wherein the rigid flex PCB assembly includes a first encoder read head connected to and extending from the second elongate segment, wherein the first encoder read head is located proximate the first end of the second elongate segment.
3. The robotic surgical system of any of the preceding clauses, wherein the first encoder read head is connected to the second rail.
4. The robotic surgical system of any of the preceding clauses, wherein the first rail includes a first linear encoder scale extending linearly along the first rail, wherein the first encoder read head is in operative registration with the first linear encoder scale.
5. The robotic surgical system of any of the preceding clauses, wherein the rigid flex PCB assembly includes a second encoder read head connected to and extending from the fourth elongate segment, wherein the second encoder read head is located proximate the first end of the fourth elongate segment.
6. The robotic surgical system of any of the preceding clauses, wherein the second encoder read head is operatively connected to the shuttle.
7. The robotic surgical system of any of the preceding clauses, wherein the second rail includes a second linear encoder scale extending linearly along the first side of the second rail, wherein the second encoder read head is in operative registration with the second linear encoder scale.
8. The robotic surgical system of clause 1, wherein the first rail includes a longitudinally extending channel formed in the first side of the first rail, and wherein the first elongate segment of the elongate body of the rigid flex PCB assembly is disposed within the longitudinally extending channel formed in the first side of the first rail.
9. The robotic surgical system of clause 8, wherein the second rail includes a first longitudinally extending channel formed in the first side of the second rail and a second longitudinally extending channel formed in the second side of the second rail, and wherein the second elongate segment of the elongate body of the rigid flex PCB assembly is disposed within the second longitudinally extending channel formed in the second side of the second rail, and wherein the third elongate segment of the elongate body of the rigid flex PCB assembly is disposed within the first longitudinally extending channel formed in the first side of the second rail.
10. The robotic surgical system of clause 1, wherein at least one of the first end of the second elongate segment of the elongate body of the rigid flex PCB assembly or the first end of the third elongate segment of the elongate body of the rigid flex PCB assembly is secured to a distal end of the second rail.
11. A linear slide mechanism for a robotic surgical system, the linear slide mechanism comprising:
a first rail having a first side and a second side opposite the first side, the second side of the first rail being connected to the robotic surgical system;
a second rail having a first side and a second side opposite the first side, the second side of the second rail being slidably connected to the first side of the first rail so as to translate axially relative to the first rail;
a shuttle slidably connected to the first side of the second rail so as to translate axially relative to the second rail; and
a rigid flex printed circuit board (PCB) assembly including an electrically conductive elongate body, the elongate body having:
   a first elongate segment having a first end and a second end;
   a second elongate segment having a first end and a second end, the second end of the second elongate segment being connected to the second end of the first elongate segment, wherein the second elongate segment overlies the first elongate segment, wherein the first elongate segment and the second elongate segment are located between the first side of the first rail and the second side of the second rail;
   a third elongate segment having a first end and a second end, the first end of the third elongate segment being connected to the first end of the second elongate segment;
   a fourth elongate segment having a first end and a second end, the second end of the fourth elongate segment being connected to the second end of the third elongate segment, wherein the fourth elongate segment overlies the third elongate segment, wherein the third elongate segment and the fourth elongate segment are located adjacent the first side of the second rail;
   a first electrical connector connected to the first end of the first elongate segment; and
   a second electrical connector connected to the first end of the fourth elongate segment and supported on the shuttle,
   wherein the elongate body of the rigid flex PCB assembly constitutes a bi-stable member.
12. The linear slide mechanism of clause 11, wherein the rigid flex PCB assembly includes a first encoder read head connected to and extending from the second elongate segment, wherein the first encoder read head is located proximate the first end of the second elongate segment, wherein the first encoder read head is connected to the second rail.
13. The linear slide mechanism of any of clauses 11-12, wherein the rigid flex PCB assembly includes a second encoder read head connected to and extending from the fourth elongate segment, wherein the second encoder read head is located proximate the first end of the fourth elongate segment, wherein the second encoder read head is operatively connected to the shuttle.
14. The linear slide mechanism of any of clauses 11-13, wherein the first rail includes a first linear encoder scale extending linearly along the first rail, wherein the first encoder read head is in operative registration with the first linear encoder scale; and wherein the second rail includes a second linear encoder scale extending linearly along the first side of the second rail, wherein the second encoder read head is in operative registration with the second linear encoder scale.
15. The linear slide mechanism of clause 11, wherein the first rail includes a longitudinally extending channel formed in the first side of the first rail, and wherein the first elongate segment of the elongate body of the rigid flex PCB assembly is disposed within the longitudinally extending channel formed in the first side of the first rail.
16. The linear slide mechanism of clause 15, wherein the second rail includes a first longitudinally extending channel formed in the first side of the second rail and a second longitudinally extending channel formed in the second side of the second rail, wherein the second elongate segment of the elongate body of the rigid flex PCB assembly is disposed within the second longitudinally extending channel formed in the second side of the second rail.
17. The linear slide mechanism of any of clauses 15-16, wherein the third elongate segment of the elongate body of the rigid flex PCB assembly is disposed within the first longitudinally extending channel formed in the first side of the second rail.
18. The linear slide mechanism of clause 11, wherein at least one of the first end of the second elongate segment of the elongate body of the rigid flex PCB assembly or the first end of the third elongate segment of the elongate body of the rigid flex PCB assembly is secured to a distal end of the second rail.
19. A robotic surgical system, comprising:
a robotic arm; and
a linear slide mechanism coupled to the robotic arm, the linear slide mechanism including:
   a rail having a first side and a second side opposite the first side, the second side of the rail being operatively connected to the robotic arm, wherein the rail includes a linear encoder scale extending linearly along the first side of the rail;
   a shuttle slidably connected to the first side of the rail so as to translate axially relative to the rail; and
   a rigid flex printed circuit board (PCB) assembly including an electrically conductive elongate body, the elongate body having:
      a first elongate segment having a first end and a second end;
      a second elongate segment having a first end and a second end, the second end of the second elongate segment being connected to the second end of the first elongate segment, wherein the second elongate segment overlies the first elongate segment; and
      an encoder read head connected to and extending from the second elongate segment, wherein the encoder read head is located proximate the first end of the second elongate segment, wherein the encoder read head is operatively connected to the shuttle, wherein the encoder read head is in operative registration with the linear encoder scale,
      wherein the elongate body of the rigid flex PCB assembly constitutes a bi-stable member.
20. The robotic surgical system of clause 19, wherein the linear slide mechanism further comprises:
a first electrical connector connected to the first end of the first elongate segment; and
a second electrical connector connected to the first end of the second elongate segment and supported on the shuttle.

Persons skilled in the art will understand that the structures and methods specifically described herein and shown in the accompanying figures are non-limiting exemplary aspects, and that the description, disclosure, and figures should be construed merely as exemplary of particular aspects. It is to be understood, therefore, that the present disclosure is not limited to the precise aspects described, and that various other changes and modifications may be effected by one skilled in the art without departing from the scope or spirit of the disclosure. Additionally, the elements and features shown or described in connection with certain aspects may be combined with the elements and features of certain other aspects without departing from the scope of the present disclosure, and that such modifications and variations are also included within the scope of this disclosure. Accordingly, the subject matter of this disclosure is not limited by what has been particularly shown and described.

What is claimed:

1. A robotic surgical system, comprising:
a robotic arm; and
a linear slide mechanism coupled to the robotic arm, the linear slide mechanism including:
a first rail having a first side and a second side opposite the first side, the second side of the first rail being connected to the robotic arm;
a second rail having a first side and a second side opposite the first side, the second side of the second rail being slidably connected to the first side of the first rail so as to translate axially relative to the first rail;
a shuttle slidably connected to the first side of the second rail so as to translate axially relative to the second rail; and
a rigid flex printed circuit board (PCB) assembly including an electrically conductive elongate body, the elongate body having:
a first elongate segment having a first end and a second end;
a second elongate segment having a first end and a second end, the second end of the second elongate segment being connected to the second end of the first elongate segment, wherein the second elongate segment overlies the first elongate segment, wherein the first elongate segment and the second elongate segment are located between the first side of the first rail and the second side of the second rail;
a third elongate segment having a first end and a second end, the first end of the third elongate segment being connected to the first end of the second elongate segment;
a fourth elongate segment having a first end and a second end, the second end of the fourth elongate segment being connected to the second end of the third elongate segment, wherein the fourth elongate segment overlies the third elongate segment, wherein the third elongate segment and the fourth elongate segment are located adjacent the first side of the second rail;
a first electrical connector connected to the first end of the first elongate segment; and
a second electrical connector connected to the first end of the fourth elongate segment and supported on the shuttle,
wherein the elongate body of the rigid flex PCB assembly constitutes a bi-stable member.

2. The robotic surgical system of claim 1, wherein the rigid flex PCB assembly includes a first encoder read head connected to and extending from the second elongate segment, wherein the first encoder read head is located proximate the first end of the second elongate segment.

3. The robotic surgical system of claim 2, wherein the first encoder read head is connected to the second rail.

4. The robotic surgical system of claim 3, wherein the first rail includes a first linear encoder scale extending linearly along the first rail, wherein the first encoder read head is in operative registration with the first linear encoder scale.

5. The robotic surgical system of claim 4, wherein the rigid flex PCB assembly includes a second encoder read head connected to and extending from the fourth elongate segment, wherein the second encoder read head is located proximate the first end of the fourth elongate segment.

6. The robotic surgical system of claim 5, wherein the second encoder read head is operatively connected to the shuttle.

7. The robotic surgical system of claim 6, wherein the second rail includes a second linear encoder scale extending linearly along the first side of the second rail, wherein the second encoder read head is in operative registration with the second linear encoder scale.

8. The robotic surgical system of claim 1, wherein the first rail includes a longitudinally extending channel formed in the first side of the first rail, and wherein the first elongate segment of the elongate body of the rigid flex PCB assembly is disposed within the longitudinally extending channel formed in the first side of the first rail.

9. The robotic surgical system of claim 8, wherein the second rail includes a first longitudinally extending channel formed in the first side of the second rail and a second longitudinally extending channel formed in the second side of the second rail, and wherein the second elongate segment of the elongate body of the rigid flex PCB assembly is disposed within the second longitudinally extending channel formed in the second side of the second rail, and wherein the third elongate segment of the elongate body of the rigid flex PCB assembly is disposed within the first longitudinally extending channel formed in the first side of the second rail.

10. The robotic surgical system of claim 1, wherein at least one of the first end of the second elongate segment of the elongate body of the rigid flex PCB assembly or the first end of the third elongate segment of the elongate body of the rigid flex PCB assembly is secured to a distal end of the second rail.

11. A linear slide mechanism for a robotic surgical system, the linear slide mechanism comprising:
a first rail having a first side and a second side opposite the first side, the second side of the first rail being connected to the robotic surgical system;
a second rail having a first side and a second side opposite the first side, the second side of the second rail being slidably connected to the first side of the first rail so as to translate axially relative to the first rail;
a shuttle slidably connected to the first side of the second rail so as to translate axially relative to the second rail; and
a rigid flex printed circuit board (PCB) assembly including an electrically conductive elongate body, the elongate body having:
a first elongate segment having a first end and a second end;
a second elongate segment having a first end and a second end, the second end of the second elongate segment being connected to the second end of the first elongate segment, wherein the second elongate segment overlies the first elongate segment, wherein the first elongate segment and the second elongate segment are located between the first side of the first rail and the second side of the second rail;
a third elongate segment having a first end and a second end, the first end of the third elongate segment being connected to the first end of the second elongate segment;
a fourth elongate segment having a first end and a second end, the second end of the fourth elongate segment being connected to the second end of the third elongate segment, wherein the fourth elongate segment overlies the third elongate segment, wherein the third elongate segment and the fourth elongate segment are located adjacent the first side of the second rail;
a first electrical connector connected to the first end of the first elongate segment; and
a second electrical connector connected to the first end of the fourth elongate segment and supported on the shuttle,
wherein the elongate body of the rigid flex PCB assembly constitutes a bi-stable member.

12. The linear slide mechanism of claim 11, wherein the rigid flex PCB assembly includes a first encoder read head connected to and extending from the second elongate segment, wherein the first encoder read head is located proximate the first end of the second elongate segment, wherein the first encoder read head is connected to the second rail.

13. The linear slide mechanism of claim 12, wherein the rigid flex PCB assembly includes a second encoder read head connected to and extending from the fourth elongate segment, wherein the second encoder read head is located proximate the first end of the fourth elongate segment, wherein the second encoder read head is operatively connected to the shuttle.

14. The linear slide mechanism of claim 13, wherein the first rail includes a first linear encoder scale extending linearly along the first rail, wherein the first encoder read head is in operative registration with the first linear encoder scale; and wherein the second rail includes a second linear encoder scale extending linearly along the first side of the second rail, wherein the second encoder read head is in operative registration with the second linear encoder scale.

15. The linear slide mechanism of claim 11, wherein the first rail includes a longitudinally extending channel formed in the first side of the first rail, and wherein the first elongate segment of the elongate body of the rigid flex PCB assembly is disposed within the longitudinally extending channel formed in the first side of the first rail.

16. The linear slide mechanism of claim 15, wherein the second rail includes a first longitudinally extending channel formed in the first side of the second rail and a second longitudinally extending channel formed in the second side of the second rail, wherein the second elongate segment of the elongate body of the rigid flex PCB assembly is disposed within the second longitudinally extending channel formed in the second side of the second rail.

17. The linear slide mechanism of claim 16, wherein the third elongate segment of the elongate body of the rigid flex PCB assembly is disposed within the first longitudinally extending channel formed in the first side of the second rail.

18. The linear slide mechanism of claim 11, wherein at least one of the first end of the second elongate segment of the elongate body of the rigid flex PCB assembly or the first end of the third elongate segment of the elongate body of the rigid flex PCB assembly is secured to a distal end of the second rail.

19. A robotic surgical system, comprising:
a robotic arm; and
a linear slide mechanism coupled to the robotic arm, the linear slide mechanism including:
a rail having a first side and a second side opposite the first side, the second side of the rail being operatively connected to the robotic arm, wherein the rail includes a linear encoder scale extending linearly along the first side of the rail;
a shuttle slidably connected to the first side of the rail so as to translate axially relative to the rail; and
a rigid flex printed circuit board (PCB) assembly including an electrically conductive elongate body, the elongate body having:
a first elongate segment having a first end and a second end;
a second elongate segment having a first end and a second end, the second end of the second elongate segment being connected to the second end of the first elongate segment, wherein the second elongate segment overlies the first elongate segment; and
an encoder read head connected to and extending from the second elongate segment, wherein the encoder read head is located proximate the first end of the second elongate segment, wherein the encoder read head is operatively connected to the shuttle, wherein the encoder read head is in operative registration with the linear encoder scale,
wherein the elongate body of the rigid flex PCB assembly constitutes a bi-stable member.

20. The robotic surgical system of claim 19, wherein the linear slide mechanism further comprises:
a first electrical connector connected to the first end of the first elongate segment; and
a second electrical connector connected to the first end of the second elongate segment and supported on the shuttle.
